(19) United States
(12) Patent Application Publication
(10) Pub. No.: US 2026/0207559 A1
(43) Pub. Date: Jul. 23, 2026

(54) (S)-(3-(1-(1H-IMIDAZOL-4-YL)ETHYL)-2-METHYLPHENYL)METHANOL FOR TREATMENT OF AGITATION

(71) Applicant: Alceptor Therapeutics, Inc., Newport Beach, CA (US)

(72) Inventors: Daniel Walter GIL, Newport Beach, CA (US); John E. DONELLO, San Clemente, CA (US); Donald E. FRAIL, San Clemente, CA (US)

(21) Appl. No.: 19/143,672

(22) PCT Filed: Dec. 27, 2023
(86) PCT No.: PCT/US2023/086022

§ 371 Date: Jun. 26, 2025

Related U.S. Application Data
(60) Provisional application No. 63/477,361, filed on Dec. 27, 2022.

(51) Int. Cl. A61K 31/4174; A61P 25/00; A61P 25/20

(52) U.S. Cl. CPC A61K 31/4174; A61P 25/00; A61P 25/20

(57) ABSTRACT

This disclosure relates to methods of treating agitation. The methods comprise administering (S)-(3-(1-(1H-imidazol-4-yl)ethyl)-2-methylphenyl) methanol to a mammal in need thereof.

### CROSS-REFERENCE TO RELATED APPLICATIONS

This application claims the benefit of U.S. Provisional Application No. 63/477,361, filed Dec. 27, 2022, which is incorporated by reference herein in its entirety.

### SUMMARY

This disclosure relates to methods of treating agitation. The methods comprise administering (S)-(3-(1-(1H-imidazol-4-yl)ethyl)-2-methylphenyl) methanol to a mammal in need thereof.

### DETAILED DESCRIPTION

Agitation is a common behavioral symptom in neurodegenerative and neuropsychiatric disorders. Disclosed herein are methods of treating agitation. These methods comprise administering (S)-(3-(1-(1H-imidazol-4-yl)ethyl)-2-methylphenyl) methanol to a mammal in need thereof.

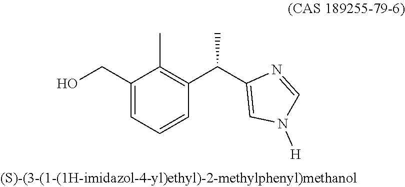 [SMILES: Cc1c(CO)cccc1[C@H](C)c1c[nH]cn1]

The terms “treating” or “treatment” broadly includes any kind of treatment activity, including the diagnosis, cure, mitigation, or prevention of disease in man or other animals, or any activity that otherwise affects the structure or any function of the body of man or other animals. In some embodiments, the mammal being treated is a human being. In some embodiments, the mammal being treated is a non-human mammal, such as a dog, a cat, a mouse, a rat, a rabbit, a monkey, a horse, a pig, etc.

For treatment of agitation and other conditions, (S)-(3-(1-(1H-imidazol-4-yl)ethyl)-2-methylphenyl) methanol may be administered orally, including by swallowing or sublingually (including buccal). Other potential routes may include subcutaneous injection, intramuscular, intravenous, etc. In some embodiments, (S)-(3-(1-(1H-imidazol-4-yl)ethyl)-2-methylphenyl) methanol is administered orally.

Unless otherwise indicated, any reference to a compound herein, such as(S)-(3-(1-(1H-imidazol-4-yl)ethyl)-2-methylphenyl) methanol, by structure, name, or any other means, includes pharmaceutically acceptable salts; alternate solid forms, such as polymorphs, solvates, hydrates, etc.; tautomers; or any other chemical species that may rapidly convert to a compound described herein under conditions in which the compounds are used as described herein.

Dosage forms, such as solid dosage forms, e.g., solid oral dosage forms, including capsules, tablets, or pills, for oral administration may also contain one or more of the following: a binder such as gum tragacanth, acacia, corn starch, or gelatin; an excipient, such as dicalcium phosphate; a disintegrating agent such as corn starch, potato starch, alginic acid, and the like; a sweetening agent such as sucrose, lactose, or saccharin; a lubricant, such as magnesium stearate; a buffer, such as a phosphate, carbonate, and/or citrate buffer; or a flavoring agent such as peppermint, oil of wintergreen, or cherry flavoring. When the dosage form is a capsule, it may contain, in addition to materials of the above type, a liquid carrier. Various other materials may be present as a coating, for example, tablets, pills, or capsules may be coated with shellac, sugar, or both. It may be desirable for material in a dosage form or pharmaceutical composition to be pharmaceutically pure and nontoxic in the amounts employed.

An effective dose of (S)-(3-(1-(1H-imidazol-4-yl)ethyl)-2-methylphenyl) methanol may vary depending upon the severity of the disease, the age of the patient, the weight of the patient, the patient's general health, the route of administration, and similar factors. In some embodiments, a human dose is about 0.01-50 mg/kg/day.

It has been found that (S)-(3-(1-(1H-imidazol-4-yl)ethyl)-2-methylphenyl) methanol has a 2- to 4-fold longer half-life than dexmedetomidine. Thus, (S)-(3-(1-(1H-imidazol-4-yl)ethyl)-2-methylphenyl) methanol may be administered less frequently than dexmedetomidine, such as once or twice a day.

Agitation may be associated with neurodegenerative conditions, such as Alzheimer disease, frontotemporal dementia (FTD), dementia, dementia with Lewy bodies (DLB), Down dementia, post-traumatic stress disorder, including aggression and hyper-arousal in post-traumatic stress disorder, Parkinson's disease, vascular dementia, vascular cognitive impairment, Sundown syndrome in Alzheimer's disease or dementia, Huntington's disease, multiple sclerosis, Creutzfeldt-Jakob disease, multiple system atrophy, and progressive supranuclear palsy; senile dementia of the Alzheimer type (SDAT), concussion and concussion syndrome, etc.

Agitation may also be associated with neuropsychiatric conditions, such as schizophrenia, bipolar disorder, bipolar mania, delirium, autism, and depression, including dementia or mood disorders in subjects with major depression (e.g., stress-related major depression).

Treatment of hyper-arousal associated with acute agitation in patients with schizophrenia, bipolar disorder, or dementia is also a contemplated use of(S)-(3-(1-(1H-imidazol-4-yl)ethyl)-2-methylphenyl) methanol.

Agitation may also be associated with surgery (pre, during, post), Traumatic Brain Injury, substance abuse and/or withdrawal (e.g., associated with opioids or alcohol).

Agitation can be both acute and chronic. In dementia, agitation is often associated with restlessness and confusion and may be preceded by pacing and asking of lots of questions. It is believed that (S)-(3-(1-(1H-imidazol-4-yl)ethyl)-2-methylphenyl) methanol has the advantage of being useful for both acute agitation events and for more chronic episodes of severe restlessness.

In some embodiments, (S)-(3-(1-(1H-imidazol-4-yl)ethyl)-2-methylphenyl) methanol is used to treat agitation associated with Alzheimer disease in a mammal, such as a human being.

In some embodiments, (S)-(3-(1-(1H-imidazol-4-yl)ethyl)-2-methylphenyl) methanol is used to treat agitation associated with frontotemporal dementia (ETD) in a mammal, such as a human being.

In some embodiments, (S)-(3-(1-(1H-imidazol-4-yl)ethyl)-2-methylphenyl) methanol is used to treat agitation associated with dementia in a mammal, such as a human being.

In some embodiments, (S)-(3-(1-(1H-imidazol-4-yl)ethyl)-2-methylphenyl) methanol is used to treat agitation associated with dementia with Lewy bodies (DLB) in a mammal, such as a human being.

In some embodiments, (S)-(3-(1-(1H-imidazol-4-yl)ethyl)-2-methylphenyl) methanol is used to treat agitation associated with Down dementia in a mammal, such as a human being.

In some embodiments, (S)-(3-(1-(1H-imidazol-4-yl)ethyl)-2-methylphenyl) methanol is used to treat agitation associated with post-traumatic stress disorder in a mammal, such as a human being.

In some embodiments, (S)-(3-(1-(1H-imidazol-4-yl)ethyl)-2-methylphenyl) methanol is used to treat agitation associated with including aggression and hyper-arousal in post-traumatic stress disorder in a mammal, such as a human being.

In some embodiments, (S)-(3-(1-(1H-imidazol-4-yl)ethyl)-2-methylphenyl) methanol is used to treat agitation associated with Parkinson's disease in a mammal, such as a human being.

In some embodiments, (S)-(3-(1-(1H-imidazol-4-yl)ethyl)-2-methylphenyl) methanol is used to treat agitation associated with vascular dementia in a mammal, such as a human being.

In some embodiments, (S)-(3-(1-(1H-imidazol-4-yl)ethyl)-2-methylphenyl) methanol is used to treat agitation associated with vascular cognitive impairment in a mammal, such as a human being.

In some embodiments, (S)-(3-(1-(1H-imidazol-4-yl)ethyl)-2-methylphenyl) methanol is used to treat agitation associated with Sundown syndrome in Alzheimer's disease or dementia in a mammal, such as a human being.

In some embodiments, (S)-(3-(1-(1H-imidazol-4-yl)ethyl)-2-methylphenyl) methanol is used to treat agitation associated with Huntington's disease in a mammal, such as a human being.

In some embodiments, (S)-(3-(1-(1H-imidazol-4-yl)ethyl)-2-methylphenyl) methanol is used to treat agitation associated with multiple sclerosis in a mammal, such as a human being.

In some embodiments, (S)-(3-(1-(1H-imidazol-4-yl)ethyl)-2-methylphenyl) methanol is used to treat agitation associated with Creutzfeldt-Jakob disease in a mammal, such as a human being.

In some embodiments, (S)-(3-(1-(1H-imidazol-4-yl)ethyl)-2-methylphenyl) methanol is used to treat agitation associated with multiple system atrophy in a mammal, such as a human being.

In some embodiments, (S)-(3-(1-(1H-imidazol-4-yl)ethyl)-2-methylphenyl) methanol is used to treat agitation associated with and progressive supranuclear palsy; senile dementia of the Alzheimer type (SDAT) in a mammal, such as a human being.

In some embodiments, (S)-(3-(1-(1H-imidazol-4-yl)ethyl)-2-methylphenyl) methanol is used to treat agitation associated with concussion in a mammal, such as a human being.

In some embodiments, (S)-(3-(1-(1H-imidazol-4-yl)ethyl)-2-methylphenyl) methanol is used to treat agitation associated with concussion syndrome in a mammal, such as a human being.

In some embodiments, (S)-(3-(1-(1H-imidazol-4-yl)ethyl)-2-methylphenyl) methanol is used to treat agitation associated with schizophrenia in a mammal, such as a human being.

In some embodiments, (S)-(3-(1-(1H-imidazol-4-yl)ethyl)-2-methylphenyl) methanol is used to treat agitation associated with bipolar disorder in a mammal, such as a human being.

In some embodiments, (S)-(3-(1-(1H-imidazol-4-yl)ethyl)-2-methylphenyl) methanol is used to treat agitation associated with bipolar mania in a mammal, such as a human being.

In some embodiments, (S)-(3-(1-(1H-imidazol-4-yl)ethyl)-2-methylphenyl) methanol is used to treat agitation associated with delirium in a mammal, such as a human being.

In some embodiments, (S)-(3-(1-(1H-imidazol-4-yl)ethyl)-2-methylphenyl) methanol is used to treat agitation associated with autism in a mammal, such as a human being.

In some embodiments, (S)-(3-(1-(1H-imidazol-4-yl)ethyl)-2-methylphenyl) methanol is used to treat agitation associated with depression, including dementia or mood disorders in a mammal with major depression (e.g., stress-related major depression).

In some embodiments, (S)-(3-(1-(1H-imidazol-4-yl)ethyl)-2-methylphenyl) methanol is used to treat hyper-arousal associated with acute agitation in a mammal, such as a human being with schizophrenia.

In some embodiments, (S)-(3-(1-(1H-imidazol-4-yl)ethyl)-2-methylphenyl) methanol is used to treat hyper-arousal associated with acute agitation in a mammal, such as a human being with bipolar disorder.

In some embodiments, (S)-(3-(1-(1H-imidazol-4-yl)ethyl)-2-methylphenyl) methanol is used to treat hyper-arousal associated with acute agitation in a mammal, such as a human being with and dementia

In some embodiments, (S)-(3-(1-(1H-imidazol-4-yl)ethyl)-2-methylphenyl) methanol is used to treat agitation associated with surgery (pre, during, post) in a mammal, such as a human being.

In some embodiments, (S)-(3-(1-(1H-imidazol-4-yl)ethyl)-2-methylphenyl) methanol is used to treat agitation associated with Traumatic Brain Injury in a mammal, such as a human being.

In some embodiments, (S)-(3-(1-(1H-imidazol-4-yl)ethyl)-2-methylphenyl) methanol is used to treat agitation associated with substance abuse and/or withdrawal (e.g., opioids or alcohol) in a mammal, such as a human being.

Treatment approaches include benzodiazepines such as Xanax and antipsychotics such as Seroquel, but these classes of drugs are highly sedating. There is a need for drugs that can be used both acutely and chronically without causing significant sedation.

In the following examples, the following are demonstrated.

(S)-(3-(1-(1H-imidazol-4-yl)ethyl)-2-methylphenyl) methanol exhibits efficacy in a model of agitation/aggression in rodents.

(S)-(3-(1-(1H-imidazol-4-yl)ethyl)-2-methylphenyl) methanol shows separation of anti-agitation doses from sedative doses in rodents despite the fact that anti-agitation and sedation both require the drug to be in the CNS.

(S)-(3-(1-(1H-imidazol-4-yl)ethyl)-2-methylphenyl) methanol shows good oral bioavailability in rat and mice models.

(S)-(3-(1-(1H-imidazol-4-yl)ethyl)-2-methylphenyl) methanol shows rapid absorption from the GI tract into the blood and entry into the brain in rats and mice.

There is differentiation of two enantiomers of (3-(1-(1H-imidazol-4-yl)ethyl)-2-methylphenyl) methanol with respect to pharmacokinetic properties, particularly oral bioavailability.

Animal models suggest(S)-(3-(1-(1H-imidazol-4-yl)ethyl)-2-methylphenyl) methanol has a higher oral bioavailability in human beings than dexmedetomidine, which may be more convenient for patients because dosing may be less frequent, e.g., once or twice a day.

Rapid absorption and sustained exposure in plasma and brain in animal models may enable use for both acute and chronic agitation.

(S)-(3-(1-(1H-imidazol-4-yl)ethyl)-2-methylphenyl) methanol is less sedating in animal models, which may enable the drug to be used to reduce agitation without putting patients to sleep.

### Example 1

We demonstrate for the first time that the dexmedetomidine metabolite, (S)-(3-(1-(1H-imidazol-4-yl)ethyl)-2-methylphenyl) methanol, is orally bioavailable with a very rapid absorption from the GI tract. Groups of rodents were dosed either intravenously or orally in order to determine certain pharmacokinetic properties, including oral bioavailability and half-life. Bioavailability was determined by comparing plasma exposure following IV dosing in a [19% hydroxypropyl beta-cyclodextrin/5% DMSO in saline] formulation with plasma exposure following dosing by oral gavage in a 0.5% methyl cellulose formulation. Oral bioavailability ranged up to 51% in mice and 34% in rats. Maximum plasma concentrations were achieved in mice and rats within 10 and 17 minutes, respectively. The drug also rapidly penetrates the blood brain barrier, rapidly achieving pharmacological concentrations in the brain within 30 minutes.

(S)-(3-(1-(1H-imidazol-4-yl)ethyl)-2-methylphenyl) methanol also exhibited sustained levels in plasma and brain with less than a mean 2-fold decrease between 2 and 8 hrs. The plasma half-life following oral dosing is 4.2-7 hrs. By comparison, dexmedetomidine oral bioavailability was only 8.6% with a half-life of 2 hrs. following oral dosing in rats. Surprisingly, (R)-(3-(1-(1H-imidazol-4-yl)ethyl)-2-methylphenyl)methanol exhibited much lower oral bioavailability of 7% in rats.

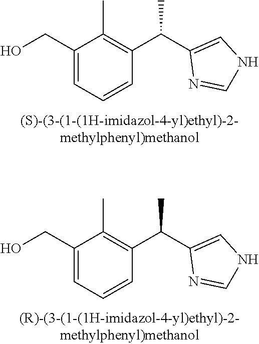 [SMILES: Cc1c(CO)cccc1[C@H](C)c1c[nH]cn1 ; Cc1c(CO)cccc1[C@@H](C)c1c[nH]cn1]

### Example 2

We also determined that (S)-(3-(1-(1H-imidazol-4-yl)ethyl)-2-methylphenyl) methanol is less susceptible than dexmedetomidine to metabolism by human recombinant cytochrome P450 (CYP) enzymes, suggesting that the longer half-life observed in rodents is likely to occur in humans as well. A 2 UM concentration of dexmedetomidine or(S)-(3-(1-(1H-imidazol-4-yl)ethyl)-2-methylphenyl) methanol were incubated for up to 60 min with 7 different human CYP enzymes and the NADPH cofactor. The % of compound remaining was measured. Five of the CYP enzymes were involved in metabolizing dexmedetomidine with 28% (CYP2B6), 84% (CYP2C8), 74% (CYP2C19), 7% (CYP2D6), and 89% (CYP3A4) remaining in the incubations. Conversely, none of the CYPs metabolized(S)-(3-(1-(1H-imidazol-4-yl)ethyl)-2-methylphenyl) methanol. A(S)-(3-(1-(1H-imidazol-4-yl)ethyl)-2-methylphenyl) methanol concentration >90% of the starting concentration was measured in all the incubations.

### Example 3

The activity of(S)-(3-(1-(1H-imidazol-4-yl)ethyl)-2-methylphenyl) methanol was tested in models of agitation and sedation.

The resident-intruder model is an established preclinical model of aggression and agitation, and allows spontaneous and natural expression of both offensive aggression/agitation and defensive behavior in laboratory rodents in a semi natural laboratory setting. When rodents are exposed to a novel male in their home cage environment, they perceive the novel male animal as an intruder and demonstrate a repertoire of defensive behaviors such as ano-genital sniffing, chasing, biting, and attacking (Nelson et al., ILAR Journal (2000) 41 (3): 153-162). The latency to attack and the number of attacks are measures of agitation. (S)-(3-(1-(1H-imidazol-4-yl)ethyl)-2-methylphenyl) methanol was effective in this model at 2 mg/kg oral and above (>80% inhibition of attacks) but demonstrated minimal activity at 1 mg/kg.

Sedation is assessed in the Locomotor Activity (LMA) model. Mice are placed in a novel chamber and their exploratory activity during a 30-minute session is measured as distance traveled. (S)-(3-(1-(1H-imidazol-4-yl)ethyl)-2-methylphenyl) methanol was partially sedating in the LMA model at 2 mg/kg (about 25% inhibition of distance travelled) and 3 mg/kg (<50% inhibition of distance travelled) and minimally sedating at 1 mg/kg.

Therefore, 2 mg/kg of(S)-(3-(1-(1H-imidazol-4-yl)ethyl)-2-methylphenyl) methanol was greater than 80% effective in a model of aggression (the resident-intruder model) at 2 mg/kg but only about 25% active in a model of sedation (the LMA model). In contrast, dexmedetomidine was greater than 80% effective in the resident-intruder model at 0.5 mg/kg but minimally effective (20% inhibition of attacks) at 0.2 mg/kg, and highly sedative (>70%) at 0.5 mg/kg and partially sedating at 0.2 mg/kg in the LMA model. The maximum achievable level of activity in the LMA model with(S)-(3-(1-(1H-imidazol-4-yl)ethyl)-2-methylphenyl) methanol was less than the maximum achievable level of activity with dexmedetomidine.

These data demonstrate that (S)-(3-(1-(1H-imidazol-4-yl)ethyl)-2-methylphenyl) methanol is advantageous over dexmedetomidine by having a larger therapeutic window between anti-agitation and sedating doses. This separation occurs even though both anti-agitation and sedation are effects that require drug activity in the brain.

If stereochemistry is not indicated, a name or structural depiction includes any stereoisomer or any mixture of stereoisomers.

Unless otherwise indicated, all numbers expressing quantities of ingredients, properties such as amounts, percentage, and so forth used in the specification and claims are to be understood in all instances as indicating both the exact values as shown and as being modified by the term “about.” Accordingly, unless indicated to the contrary, the numerical parameters set forth in the specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques. Rounding any value to fewer significant digits is specifically contemplated herein. For example, the term “about 1.1” is intended to contemplate a value of “about 1.” Similarly, a value such as “about 100” is intended to contemplate a value of “about 1×10²,” indicating only a single significant digit, and that values should be rounded so that there is only a single significant digit.

The terms “a,” “an,” “the” and similar referents used in the context of describing the embodiments (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or representative language (e.g., “such as”) provided herein is intended merely to better illuminate the embodiments and does not pose a limitation on the scope of any claim. No language in the specification should be construed as indicating any non-claimed element essential to the practice of the claims.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or to expedite prosecution. When any such inclusion or deletion occurs, the specification is deemed to contain the group as modified thus fulfilling the written description of all Markush groups if used in the appended claims.

Any use of the terms “comprising,” “comprises,” “having,” “includes,” “including,” or the like is intended to also contemplate use of “consisting essentially of,” “consists essentially of,” “consisting of,” or “consists of.”

Certain embodiments are described herein, including the best mode known to the inventors for carrying out the claimed embodiments. Of course, variations on these described embodiments will become apparent to those of ordinary skill in the art upon reading the foregoing description. The inventor expects skilled artisans to employ such variations as appropriate, and the inventors intend for the claimed embodiments to be practiced otherwise than specifically described herein. Accordingly, the claims include all modifications and equivalents of the subject matter recited in the claims as permitted by applicable law. Moreover, any combination of the above-described elements in all possible variations thereof is contemplated unless otherwise indicated herein or otherwise clearly contradicted by context.

In closing, it is to be understood that the embodiments disclosed herein are illustrative of the principles of the claims. Other modifications that may be employed are within the scope of the claims. Thus, by way of example, but not of limitation, alternative embodiments may be utilized in accordance with the teachings herein. Accordingly, the claims are not limited to embodiments precisely as shown and described.

1. A method of treating agitation comprising administering (S)-(3-(1-(1H-imidazol-4-yl)ethyl)-2-methylphenyl) methanol to a mammal in need thereof.

2. The method of claim 1, wherein the agitation is associated with a neurodegenerative condition.

3. The method of claim 1, wherein the agitation is associated with a neuropsychiatric condition.

4. The method of claim 1, wherein the agitation is associated with surgery.

5. The method of claim 1, wherein the agitation is associated with traumatic brain injury.

6. The method of claim 1, wherein the agitation is associated with substance abuse or withdrawal.

7. The method of claim 1, wherein the (S)-(3-(1-(1H-imidazol-4-yl)ethyl)-2-methylphenyl) methanol is administered orally.

8. The method of claim 1, 2, 3, 4, 5, 6, or 7, wherein the mammal is a human being.

9. The method of claim 1, wherein the (S)-(3-(1-(1H-imidazol-4-yl)ethyl)-2-methylphenyl) methanol is orally administered to the mammal.

10. The method of claim 1, 2, 3, 4, 5, 6, 7, 8, or 9, wherein the mammal experiences minimal sedation.

11. An oral dosage form comprising (S)-(3-(1-(1H-imidazol-4-yl)ethyl)-2-methylphenyl) methanol.
